(19) United States
(12) Patent Application Publication
(10) Pub. No.: US 2005/0074867 A1
(43) Pub. Date: Apr. 7, 2005

(54) Preservation of competent cells at ambient temperatures

(76) Inventors: Victor Bronshtein, San Diego, CA (US); Gordana Dlordijevic, San Diego, CA (US); Charles E. Isaac, Carlsbad, CA (US)

Correspondence Address: Leon R Yankwich;Yankwich & Associates, 201 Broadway, Cambridge, MA, 02139, US

(21) Appl. No.: 10/477,014

(22) PCT Filed: May 7, 2002
(86) PCT No.: PCT/US02/14552

Related U.S. Application Data
(60) Provisional application No. 60/289,558, filed on May 7, 2001.

(51) Int. Cl. C12N 1/20

(52) U.S. Cl. 435/252.33

(57) ABSTRACT

The present invention relates to techniques for loading cells with desiccation protectants comprising non-metabolizable and non-reducing carbohydrate analogs. Specifically, competent bacterial cells can be preserved using the loading techniques provided herein.

### BACKGROUND OF THE INVENTION

1. Field of the Invention

The present invention relates to loading techniques which can be used in conjunction with biological preservation systems. Specifically, competent cells can be preserved using the loading techniques provided herein.

2. Description of the Related Art

Routine procedures in biotechnological laboratories involve daily use of various competent cells for cloning, propagation and preparation of plasmid DNA, construction of genomic libraries, protein expression, and mutagenesis. Different kinds of competent cells are available commercially, including bacterial, insect, yeast and mammalian cell lines. Various strains of the gram-negative bacterium Escherichia coli (E coli) are one of the most extensively used competent cells.

Depending on the method of transformation that will be applied, competent cells can be prepared as chemically competent (to be transformed by a heat pulse) or as electrocompetent (to be transformed by an electrical pulse). Chemically competent cells are readily transformable only in the early logarithmic growth stage. However, early logarithmic cells are extremely sensitive to various stresses, including osmotic and thermal stress, making their preservation difficult. As an example, preservation survival of <1% in dried logarithmic E. coli cells was recently reported by D. Billi et al. (Billi, D., D. J. Wright, R. F. Helm, T. Prickett, M. Potts, and J. H. Crowe. 2000. Engineering desiccation tolerance in Escherichia coli. Appl. Environ. Microbiol. 66:1680-1684). In contrast to chemically competent cells, electrocompetent cells can be transformed in later logarithmic stage and are, therefore, more amenable to preservation.

Regardless of the nature of the competent cells and their final use, the cells must be constantly held at subzero temperatures. To allow efficient use, competent cells are often prepared in large quantities and then preserved and stored. Present methodology for the preservation of competent cells includes mixing with different cryoprotectants, for example glycerol, sucrose, dimethly sulfoxide (DMSO), and freezing at −80° C. Because presently available preservation methods include freezing at −80° C., competent cells are routinely subjected to damage by osmotic stress and by exposure to freezing.

Commercially available competent cells are sold individually or as components of various kits developed in order to improve efficiency and accuracy of many routine procedures conducted in research laboratories. Most of the kit components, except competent cells, can be stored in laboratory refrigerators or at room temperature. Therefore, the necessity to maintain competent cells at sub-zero temperatures imposes significant burden on distribution of the material and its subsequent storage and use (significant laboratory freezer space is often allocated for the storage of competent cells). Also, the need for sub-zero temperatures prevents use of competent cells and kits containing the cells in various facilities where freezers are not available. Therefore, the demand for a technology that will alleviate the need for handling and storing of competent cells at sub-zero temperatures is certainly present.

### SUMMARY OF THE INVENTION

The present invention discloses loading techniques which can be used in conjunction with biological preservation systems. Specifically, methods for preserving competent cells, such as E. coli, at ambient temperatures are disclosed. Loading techniques described herein increase desiccation tolerance which allows preservation by foam formation.

In one embodiment of the present invention, a method for preserving competent cells for storage at ambient temperatures is disclosed. In the methods described therein, cells are incubated with sugar solution and dried by foam formation. The sugar solution used can be any of a-methyl-glucoside (MAG), 2-deoxyglucose (2-DOG), sucrose, raffinose, or glucose. In a preferred embodiment, cells contemplated for preservation by the present invention include gram negative bacteria and, specifically, E. coli.

In another embodiment of the present invention, cells may be electrocompetent or chemically competent. In a preferred embodiment, competence of chemically competent cells is achieved by mixing cells with CaCl₂ or RbCl.

In a further embodiment of the present invention, the preserved cells of the present invention may be rehydrated. In a preferred embodiment, the rehydration solution may consist of carbohydrates, mono-valent cations, divalent cations, organic buffers, and water. In an especially preferred embodiment, sucrose is a preferred carbohydrate. Additionally, calcium and rubidium are preferred cations.

In another aspect of the invention, methods which enhance desiccation tolerance in logarithmic cells are disclosed. In the methods described therein, cells are incubated with nonmetabolizable and non-reducing carbohydrate analogs. Preferred analogs of the invention include MAG or 2-DOG.

In a further embodiment of the invention, the carbohydrate analog is administered at concentrations of 0.1%-50% of the total preservation solution. In an especially preferred embodiment of the invention, the carbohydrate analog is administered at concentrations of 5%-15% of the total preservation solution.

The logarithmic cells of the invention are mid or late logarithmic cells. Furthermore, in a preferred embodiment, the logarithmic cells are harvested at OD₅₅₀ between 0.1-2.0. In another embodiment, the logarithmic cells are harvested at OD₅₅₀ between 0.3-1.0. In an especially preferred embodiment, the logarithmic cells are harvested at OD₅₅₀ at 0.5.

In a further embodiment of the invention, incubation is conducted at 0° C.-60° C. In another embodiment of the invention, incubation is conducted at 20° C.-40° C. In an especially preferred embodiment of the invention, incubation is conducted at 30° C.-40° C. Furthermore, in another aspect of the invention, incubation is conducted for 0-60 minutes. In another aspect of the invention, incubation is conducted for 2.5-60 minutes. In an especially preferred embodiment, incubation is conducted for 30-60 minutes.

In another aspect of the invention, the carbohydrate analogs of the invention are actively transported into logarithmic cells. Additionally, logarithmic cells may be grown in the presence of glucose prior to preservation to prime cells for active transport. The growth solution of the invention may contain 0.001%-50% glucose. In an especially preferred embodiment of the invention, the growth solution may contain 0.05%-5% glucose.

According to the present invention, preservation yield of cells dried by foam formation may be increased by incubating cells with sugar solution prior to subjecting the cells to preservation conditions.

### BRIEF DESCRIPTION OF THE DRAWINGS

FIG. 1 is a bar chart showing preservation survival of electrocompetent E. coli cells preserved by foam formation.

FIG. 2 is a bar chart showing electroporation survival of electrocompetent E. coli cells preserved by foam formation.

FIG. 3 is a bar chart showing electroporation efficiency of electrocompetent E. coli cells preserved by foam formation.

FIG. 4 is a line graph showing the effect of increasing concentration of glucose analogs during in vitro loading on preservation survival of logarithmic E. coli.

FIG. 5 is a line graph showing the effect of loading temperature on preservation survival of E. coli.

FIG. 6 is a line graph showing the effect of loading temperature on stability of the preserved E. coli.

FIG. 7 is a line graph showing the effect of loading time on preservation survival and stability of E. coli cells loaded with 10% MAG at 37° C.

FIG. 8 is a line graph showing the effect of growth stage on the preservation survival of E. coli loaded with 10% MAG.

### DETAILED DESCRIPTION OF THE PREFERRED EMBODIMENT

The present invention discloses loading techniques which can be used in conjunction with biological preservation systems. Specifically, the invention relates to methods of using loading techniques to increase desiccation tolerance that will allow preservation by foam formation. In a preferred embodiment, the methods described herein may be used to preserve E. coli cells.

In order to preserve competent cells under conditions less damaging then freezing and to allow their subsequent handling at temperatures above sub-zero, we used the foam formation process. Foam formation technology represents an advanced method for preservation by drying, where the sample temperature is always kept above freezing (U.S. Pat. No. 5,766,520). Therefore, the possibility for cell damage by formation of ice-crystals, which is the most common damage during preservation by freezing, is eliminated. However, because drying itself imposes significant osmotic stress on logarithmic cells, cellular desiccation tolerance should be optimized in order to take the full advantage of the foam formation preservation process. We demonstrated that incubation of the cells with an appropriate sugar solution prior to preservation (“loading”), can significantly increase preservation yield of cells dried by foam formation.

Herein we describe a method to preserve E coli cells at ambient temperature by the foam formation process and demonstrate that the preserved cells are transformable at efficiency comparable to that of the fresh cells.

Preservation of Electrocompetent Cells

Preservation of electrocompetent cells in the various working examples included infra were conducted as follows, unless otherwise stated:

(1) Preparation of fresh electrocompetent cells: Escherichia coli ATCC strain MM294 cells were grown under standard conditions in L-broth (growth medium for E. coli), at 37° C., with moderate aeration (shaking at 250 rpm). An isolated colony from a fresh streak was inoculated into 5 ml of L-broth and incubated overnight (preculture). The following morning, the preculture was subcultured (1% inoculum) in 100 ml of L-broth+glucose (0.05%-0.5%). The cells were incubated until OD₅₅₀=0.5 (subculture). At this point, the cells were transferred into 250 ml of L-broth+glucose (0.05%-0.5%) and incubation continued until OD₅₅₀=0.8. Two flasks with 250 ml of cells were prepared.

When the culture OD₅₅₀ reached 0.8 (cell density approx. 5×10⁸ cfu/ml), the cells were harvested by centrifugation at 2,500 rpm (at room temperature) for 10 minutes. The cells (2×200 ml samples) were washed once with an equal volume of PBS buffer or twice with an equal volume of 20% ice-cold sucrose and centrifuged again as described above. The cells were concentrated 100-fold in PBS buffer or in 20% sucrose to cell density of approx. 2×10¹⁰ cfu/ml. Bacterial counts were determined by diluting cells in PBS buffer and spread plating appropriate dilutions in duplicate on L-agar. The plates were incubated at 37° C. overnight and colonies were counted the following day.

(2) Electroporation of fresh E. coli electrocompetent cells: Two aliquots of the concentrated cells (50 μl each) were transferred into pre-chilled Eppendorf tubes (1.5 ml) and 2 μl of pUC18 plasmid DNA (approx. 300 ng/μl) were added to the tubes. The [cells+DNA] mixtures were incubated on ice for 5 minutes and transferred into pre-chilled cuvettes for electroporation. The cells were electroporated by using a BTX T-100 electroporator, at 625 or at 800 volts (V) in BTX cuvettes (0.1 cm gap, P/N 610 Disposable Cuvettes) with a time constant “r” equal 5 msec. Under the pulse conditions described above, the cells were subjected to an electrical field “E” equal to 6.25 kV/cm or 8.0 kV/cm.

Immediately after the pulse, the cells were transferred into small glass tubes (10×100 mm) with 0.5 ml of “outgrowth medium” (L-broth+20% sucrose+10 mM CaCl₂). The electroporated bacteria were incubated at 37° C. with moderate shaking (150 rpm) for 60 minutes. When the 60 minutes period elapsed, the cells were diluted in “outgrowth medium” and duplicate samples were spread plated on L-agar plates with no antibiotic (to determine electroporation survival) and on L-agar plates with 50 μg/ml or 100 μg/ml of ampicillin (selective marker on pUC18 plasmid; to determine electroporation efficiency). The plates were incubated overnight at 37° C. and the colonies were counted the next day.

(3) Loading of fresh electrocompetent E. coli cells before preservation: When applicable, one aliquot of the cell concentrate (3.2 ml of cells in PBS buffer, approx. 2×10¹⁰ cfu/ml) was mixed with 0.8 ml of 50% methyl-glucoside (MAG; 10% final concentration) and incubated at 37° C. for 30 minutes (in vitro loading). A second aliquot (3.2 ml of the cell concentrate) was mixed with 0.8 ml of PBS buffer and incubated on ice for 30 minutes (control, unloaded cells). Similar to loading with MAG, the cells were loaded with 2-deoxyglucose (2-DOG) as described in Example 5 infra.

(4) Preservation of electrocompetent E. coli cells: The cells (resuspended in PBS buffer or on 20% sucrose) were mixed with an equal mass of preservation solution consisting of 45% 2:1 sucrose:raffinose or 45% 4:1 sucrose:MAG on ice. Altematively, the cell pellets were resuspended directly in 22.5% sucrose:raffinose or 22.5% 4:1 sucrose:MAG. The 0.5 g aliquots were distributed in 5 ml glass serum vials or 0.2 g aliquots were distributed in 1.2 ml glass serum vials. To determine the survival in preservation solution before drying, cell-containing mixtures were diluted in PBS buffer containing 20% sucrose and appropriate dilutions were spread plated in duplicate on L-agar plates.

The samples were dried by foam formation. The mixtures were foamed at 0° C. and kept at that temperature until foam formation occurred. After foam was well formed, shelf temperature was increased to 20° C. and drying continued overnight (at least 12 hours).

When applicable, electrocompetent cells were frozen in the following manner. The cell suspension (100 μl) was mixed with 10% DMSO (10 μl) and slowly frozen at −80° C.

(5) Assaying the preserved E. coli cells: Dried samples were rehydrated at room temperature. 1 ml of rehydration solution (20% sucrose) was added to the 5 ml vials and 0.2 ml was added to the 1.2 ml vials. For the determination of preservation survival, rehydrated cells were diluted and spread plated in duplicate on L-agar plates. Plates were incubated at 37° C. overnight and colonies were counted the following day.

(6) Electroporation of dried (preserved) E. coli cells: One or two 50 μl aliquots were removed from each rehydrated vial and transferred to pre-chilled Eppendorf tubes. The cells were mixed with pUC18 plasmid DNA (300 ng/μl) and electroporated using the conditions described previously for the electroporation of fresh electrocompetent cells.

Electroporation survival was calculated as the number of viable cells recovered after electrical pulse per total number of electroportated cells. Electroporation efficiency was calculated as the number of transformants (cells which received pUC18 plasmid DNA) per number of cells recovered after electroporation (electroporation survival). When applicable, the electroporation efficiency was also expressed as number of transformants per 1 μg of pUC18 DNA (Cfu/μg).

Preservation of Chemically Competent Cells

Preservation of chemically competent cells in the various working examples included infra was conducted as follows, unless otherwise stated:
(1) Preparation of chemically competent cells: The cells were grown as described for preparation of electrocompetent cells, except that the culture was harvested when OD₅₅₀ reached 0.5. The cells were chilled on ice and centrifuged as described for electrocompetent cells. The supernatant was removed and the cells were and re-suspend in 30 ml of ice-cold Buffer A [30 mM KOAc, 50 mM MnCl₂, 100 mM RbCl, 10 mM CaCl₂, 10% sucrose), pH=5.8. The cells were incubated on ice for 120 minutes. When the 120 minutes period elapsed, the cells were centrifuged for 5 minutes at 2,500 rpm in a pre-chilled centrifuge. The supernatant was removed and the cells resuspended in 4 ml of ice-cold Buffer B [10 mM Na MOPS (pH=7.0), 75 mM CaCl₂, 10 mM RbCl, 10% sucrose]. The cells were kept on ice at all times.
(2) Preservation of chemically competent E. coli cells: The cells were transferred into ice-cold 15 ml Falcon tubes. 3.5 ml of cells were mixed with 3.5 g of ice-cold preservation solution consisting of 40% 3:1 sucrose:MAG in PBS buffer. The 2×0.1 ml of preservation mixture were removed, serially diluted with PBS buffer and plated in duplicate on agar plates with no antibiotic (“preservation mixture controls”). The 0.5 g aliquots of preservation mixture were distributed into pre-chilled 5 ml sterile glass vials. The mixtures were preserved by foam formation as described for drying of electrocompetent cells.
(3) Assaying of the preserved chemically competent E. coli cells: Two vials of the dried cells were transferred on ice and re-hydrated with 0.5 ml of Buffer B. For the determination of preservation survival, rehydrated cells were diluted in buffer B and spread plated in duplicate on L-agar plates. Plates were incubated at 37° C. overnight and colonies were counted the following day. The vials were stored in a refrigerator for future assays.
(4) Transformation of chemically competent E. coli cells by “heat shock”: The fresh and the preserved cells were transformed under identical conditions, except that in order to keep the same cell density during the transformation, 0.2 ml of the preserved cells and 0.1 ml of the fresh cells were used. The cells were transformed in the following manner. 0.1 ml of the fresh cells or 0.2 ml of re-hydrated content from each vial were transferred into pre-chilled Eppendorf tubes. The pUC18 plasmid DNA (5 μl, approx. 500 ng) was added into each Eppendorf tube with cells. The cells and DNA were mixed gently and the mixtures were incubated on ice for 120 minutes. When the 120 minutes period elapsed, the cells were transferred for 90 seconds at 42° C. (“heat shock”). The cells were returned on ice for 2 minutes and then transferred into 5 ml glass tubes containing 5 volumes of L-broth pre-warmed at room temperature. The cells were shaken gently at 37° C. for 60 minutes (150 rpm). Following the 60 minutes incubation at 37° C., 0.1 ml aliquots of the cells were removed from the tubes, diluted with L-broth medium and spread-plated in duplicate on L-agar plates with 50 μg/ml of ampiclin (Ap₅₀). The plates were incubated aerobically at 37° C. overnight.

The following working examples illustrate preservation of electrocompetent and chemically competent cells by the use of loading in accordance with the methods of the present invention:

### EXAMPLE 1

Electrocompetent E. coli MM294 cells were prepared as described previously, except for as follows:

In this experiment, the cells were washed twice in ice-cold 20% sucrose and concentrated 100-fold after the second wash. Additionally, four samples were prepared (A, B, C, and D). The cell concentrates were diluted 1:1 with the preservation solution consisting of 45% 2:1 sucrose:raffinose (sample A) or resuspended directly in 22.5% 2:1 sucrose:raffinose (samples B and C). Samples A and B were preserved in 0.5 ml vials (0.5 g fill per vial). Sample C was preserved in 1.2 ml vials (0.2 g fill per vial). Additional solution consisting of 22.5% 4:1 sucrose:MAG was prepared and evaluated for preservation of the cells (sample D). The cell concentrate (“D”) was resuspended directly in 22.5% 4:1 sucrose:MAG and preserved in 1.2 ml vials (0.2 g fill per vial). Dried cells of all samples were rehydrated in 20% sucrose and assayed for stability (1 ml was added to the cells in 5 ml vials, 0.2 ml to the cells in 1.2 ml vials). The preserved cells of sample B were rehydrated with nano-pure water (0% sucrose), 10%, and 20% sucrose and electroporated. Electroporation survival and efficiencies in samples rehydrated with solutions containing different amounts of sucrose were determined. Finally, fresh and dried cells were electroporated at 800V. Electroporation survival and efficiencies were determined and compared.

Preservation yield, electroporation survival and efficiencies obtained with the cells in four different samples are shown in Table 1.

TABLE 1

| | | | |
|---|---|---|---|
| Sample | Preservation | Electroporation | Electroporation |
| Description | Survival (%) | Survival (%) | Efficiency (10⁻³%) |

Fresh Cells

| | | |
|---|---|---|
| ND | 95.8 +/− 17.2 | 0.50 +/− 0.06 |

Foam Formation Preserved (Day 0)

| | | | |
|---|---|---|---|
| A | 36.8 +/− 2.7 | 36.6 +/− 3.9 | 8.1 +/− 0.8 |
| B | 21.5 +/− 5.6 | 70.2 +/− 12.4 | 2.2 +/− 0.5 |
| C | 18.5 +/− 3.2 | 43.0 +/− 8.1 | 2.7 +/− 0.2 |
| D | 18.7 +/− 2.9 | 63.9 +/− 11.4 | 3.7 +/− 0.2 |

Foam Formation Preserved (Day 11 at 4° C.)

| | | | |
|---|---|---|---|
| A | 39.8 +/− 4.5 | 15.1 +/− 1.7 | 19.8 +/− 2.5 |
| B | 30.2 +/− 3.2 | 19.9 +/− 7.7 | 3.3 +/− 1.7 |
| C | 22.7 +/− 5.8 | 19.1 +/− 2.4 | 3.7 +/− 0.9 |
| D | 32.2 +/− 7.8 | 13.8 +/− 1.0 | 10.4 +/− 0.9 |

Foam Formation Preserved (Day 11 at RT°)

| | | | |
|---|---|---|---|
| A | 36.2 +/− 6.4 | 19.5 +/− 1.5 | 21.1 +/− 5.1 |
| B | 25.9 +/− 1.5 | 25.5 +/− 5.9 | 4.7 +/− 1.1 |
| C | 23.6 +/− 8.6 | 10.1 +/− 5.6 | 8.2 +/− 1.6 |
| D | 27.6 +/− 6.5 | 15.0 +/− 6.6 | 8.2 +/− 1.9 |

Foam Formation Preserved (Day 20 at 4° C.)

| | | | |
|---|---|---|---|
| A | 38.9 +/− 10.9 | 30.6 +/− 2.0 | 17.2 +/− 5.5 |
| B | N/D | N/D | N/D |
| C | N/D | N/D | N/D |
| D | N/D | N/D | N/D |

Foam Formation Preserved (Day 20 at RT°)

| | | | |
|---|---|---|---|
| A | 30.3 +/− 2.3 | 33.9 +/− 2.9 | 22.8 +/− 8.2 |
| B | 17.8 +/− 5.3 | 22.3 +/− 3.5 | 5.6 +/− 3.1 |
| C | 27.4 +/− 10.4 | 18.2 +/− 3.6 | 5.9 +/− 0.8 |
| D | 21.7 +/− 0.5 | 29.9 +/− 6.7 | 5.1 +/− 0.8 |

The cell concentrate diluted 1:1 in the preservation solution (A) preserved at a higher yield compared to the concentrate (B) resuspended directly in preservation solution (36.8% versus 21.5%). There was no significant difference in the preservation yield or stability of the preserved samples with respect to the preservation solution used (18.5% in sample C and 18.7% in sample D at Day 0). Regardless of the concentration of the cells in a sample or a vial size, the preserved cells were stable for 20 days at RT and at 4° C.

Comparison of the electroporation parameters in preserved and fresh cells revealed that electroporation survival in the preserved cells was lower than in the fresh cells. In contrast, electroporation efficiency in the preserved cells was higher. The preserved cells routinely electroporated at the efficiency 3 to 10 fold higher than the fresh cells.

With respect to electroporation parameters in the preserved cells, electroporation survival at Day 0 was higher when the cells were more concentrated (sample B versus sample A). When the cells were preserved in small aliquots (0.2 g in 1.2 ml vial), electroporation survival was higher in the cells preserved in sucrose:MAG (D) compared to the cells preserved in sucrose:raffinose (C). Differences in electroporation survival between different samples were smaller at Day 20 than at Day 0.

At Day 0, electroporation efficiency was somewhat higher in the preserved cells in sample A than in sample B. Similar to electroporation survival, the electroporation efficiency was slightly higher at Day 0 in the cells preserved in sucrose:MAG compared to the cells preserved in sucrose:raffinose. At all times (Day 0-Day 20), transformation efficiency was significantly higher in the preserved cells in sample A compared to the cells in other samples.

The effect of the sucrose concentration in the rehydration medium on preservation survival and electroporation parameters was evaluated. Relevant data are presented in Table 2.

TABLE 2
Preserved cells of sample B rehydrated
with different amounts of sucrose

| | Preservation | Electroporation | Electroporation |
|---|---|---|---|
| Sample Description | Survival (%) | Survival (%) | Efficiency (10⁻³%) |
| 0% sucrose | 14.8 +/− 2.9 | 37.3 +/− 6.3 | 45.6 +/− 1.5 |
| 10% sucrose | 32.4 +/− 8.0 | 35.4 +/− 4.0 | 10.7 +/− 1.4 |
| 20% sucrose | 23.4 +/− 2.8 | 74.5 +/− 8.2 | 0.9 +/− 0.3 |

According to data presented in Table 2, the cells rehydrated without sucrose had lower preservation yield than the samples rehydrated with 10% or 20% sucrose (14.8% versus 23-32%). Similar to the preservation survival, the electroporation survival was higher in the preserved cells rehydrated in the solution containing the higher concentration of sucrose (74.5% in 20% sucrose versus 37.3% with no sucrose).

Increase in the amount of sucrose in the rehydration solution had an inhibitory effect on electroporation efficiency. The efficiency in the cells rehydrated without sucrose was 45.6% compared to 0.9% in the cells rehydrated with 20% sucrose (Table 2).

Based on the data from this example, the following conclusions were reached:
1. Electrocompetent E. coli cells could be successfully preserved by foam formation.
2. There was no difference in preservation survival when the cells were preserved in small aliquots (0.2 g in 1.2 ml vial compared to 0.5 g in 0.5 ml vial).
3. Preservation survival and stability were slightly better in diluted cells (A versus B).
4. There was no significant difference in preservation survival when the cells were preserved in sucrose:raffinose or in sucrose:MAG.
5. Electroporation survival in the preserved cells was lower than in the fresh cells.
6. Electroporation efficiency in the preserved cells was routinely higher than in the fresh cells. Efficiency was the highest in the cells which were diluted 1:1 with preservation solution.
7. Increase in the amount of sucrose in rehydration medium enhanced preservation and electroporation survival, but was inhibitory with respect to the electroporation efficiency.

### EXAMPLE 2

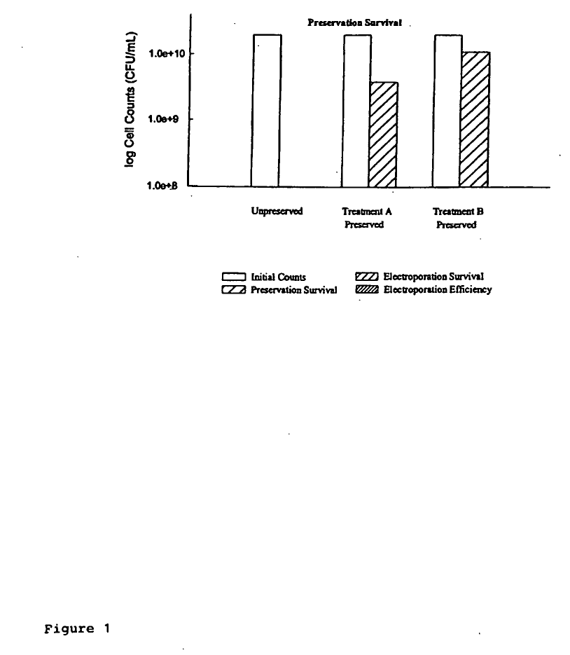

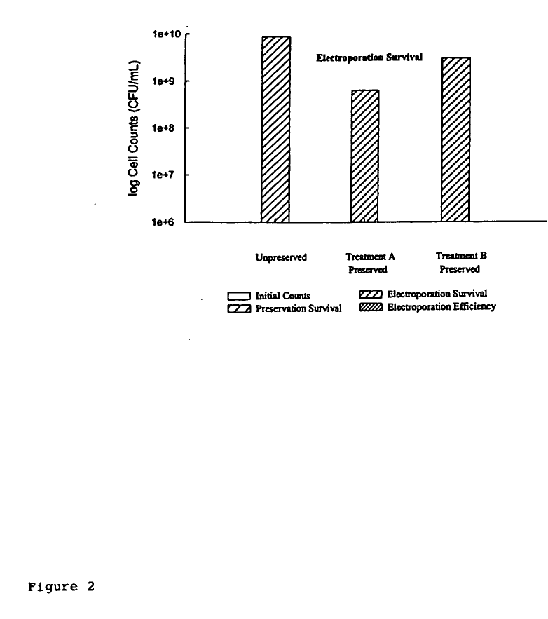

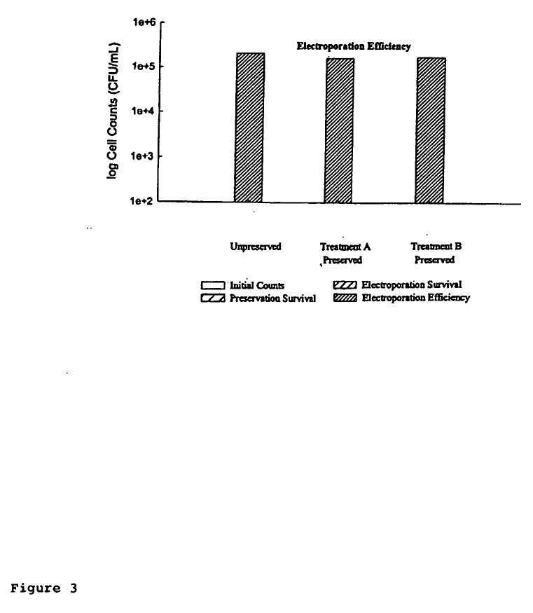

To enhance bacterial desiccation tolerance, the cells were loaded with 10% MAG. The yield of the preserved electrocompetent E. coli cells, electroporation survival and electroporation efficiency of fresh and preserved cells (loaded and unloaded control) were evaluated and compared (Table 3, FIG. 1, FIG. 2, and FIG. 3).

TABLE 3

| | | | Electroporation | | Electroporation | |
|---|---|---|---|---|---|---|
| | Preservation Yield | | Survival | | Efficiency | |
| Cells | Cfu/ml | % | Cfu/ml | % | Cfu/ml | 10⁻³% |
| Fresh | 2.0 × 10¹⁰ | N/A | 8.6 × 10⁹ | 43.5 | 2.1 × 10⁵ | 2.4 |
| Preserved (Dried) | | | | | | |
| Control | 3.8 × 10⁹ | 19.3 | 6.3 × 10⁸ | 16.5 | 1.6 × 10⁵ | 25.4 |
| (Treatment A) | | | | | | |
| Loaded | 1.1 × 10¹⁰ | 53.5 | 3.0 × 10⁹ | 28.6 | 1.7 × 10⁵ | 5.6 |
| (Treatment B) | | | | | | |

N/A, not applicable.
The data represents the average of three independent experiments.

The preservation yield of the loaded dried electrocompetent cells was significantly higher than the yield in unloaded controls (53.5% versus 19.3%, Table 3). Therefore, loading with MAG ameliorated desiccation damage during foam formation resulting in increased preservation survival.

Electroporation survival of dried cells was lower than in fresh cells (Table 3). In dried loaded cells, electroporation survival was higher than in dried control (28.6% versus 16.5%, Table 3). Electroporation efficiency of the preserved loaded cells was somewhat higher than in the fresh cells (5.6×10⁻³% versus 2.4×10⁻³%, Table 3).

The following conclusions were made based on the data from this example:
1. Loading of the cells with 10% MAG significantly increased preservation survival.
2. Electroporation survival in the preserved cells was lower than in the fresh cells.
3. Similar to preservation survival, loading of the cells with 10% MAG significantly increased electroporation survival.
4. Electroporation efficiency (cfu/ml) in the preserved cells was comparable or slightly higher compared to the fresh cells.

### EXAMPLE 3

In this example, commercial electrocompetent E. coli cells, in the form of a bacterial pellet, were preserved by the foam formation process. The cells were concentrated 100-fold and preserved as described previously. To increase desiccation tolerance and enhance preservation survival. One aliquot of the cells were loaded in vitro with 10% MAG. The preserved cells were stored at 4° C. In addition to preservation by foam formation, the cells were preserved by slow freezing (with 10% DMSO) at −80° C.

Preservation yields and stability in commercial electrocompetent E. coli cells preserved by foam formation and by freezing are presented in Table 4.

TABLE 4

| | Preservation yield** | | Stability (12 days) | |
|---|---|---|---|---|
| Cells | Cfu/ml | % | Cfu/ml | % |
| Fresh | 1.4 × 10¹⁰ +/− 2.8 × 10⁹ | 100 +/− 19.9 | N/A | N/A |
| Frozen* | 1.1 × 10¹⁰ +/− 2.1 × 10⁹ | 75.6 +/− 14.8 | 1.2 × 10¹⁰ +/− 5.4 × 10⁸ | 82.7 +/− 3.9 |
| Preserved (Control)* | 6.6 × 10⁹ +/− 4.5 × 10⁸ | 47.7 +/− 3.2 | 4.9 × 10⁹ +/− 1.5 × 10⁹ | 35.8 +/− 10.9 |
| Preserved (Loaded) | 8.8 × 10⁹ +/− 3.9 × 10⁸ | 63.6 +/− 2.8 | 9.5 × 10⁹ +/− 1.1 × 10⁸ | 68.5 +/− 0.8 |

*The control cells were also assayed for stability after 67 days at 4° C. Some loss in viability was observed (20 +/− 1% survival, 3 × 10⁹ +/− 2.0 × 10⁸ cfu/ml).
**Frozen cells were incubated 72 hours at −80° C.; Foam formation-preserved cells were dried overnight.
N/A, not applicable.

The control (unloaded) commercial cells dried by foam formation were preserved at a 47.7% yield. The cells were relatively stable at 4° C. (35.8% viability after 12 days and 20% viability after 67 days storage). Commercial cells loaded in vitro with 10% MAG and preserved by foam formation had a yield of 63.6%. These cells were completely stable at 4° C. (68.5% yield after 12 days). The frozen cells were also stable after 12 days of storage at −80° C.

Preserved commercial cells (vitrified and frozen) were electroporated as described previously. Electroporation survival and electroporation efficiency in the preserved cells were determined and compared to the same parameters in the fresh cells (Table 5).

TABLE 5

| | | | |
|---|---|---|---|
| | | *Preserved | *Preserved |
| Cells | Fresh | (Control) | (Loaded) |

Electroporation Survival

| | | | |
|---|---|---|---|
| (Cfu/ml) | 6.40 × 10⁹ +/− 1.3 × 10⁹ | 7.9 × 10⁸ +/− 1.2 × 10⁸ | 3.2 × 10⁹ +/− 4.2 × 10⁸ |
| (%) | 46.2 +/− 9.4 | 12.2 +/− 1.7 | 35.7 +/− 4.7 |

Electroporation Efficiency

| | | | |
|---|---|---|---|
| (Cfu/ml) | 1.5 × 10⁵ +/− 4.0 × 10⁴ | 7.0 × 10⁴ +/− 5.1 × 10⁴ | 1.9 × 10⁵ +/− 1.7 × 10⁵ |
| (10⁻³%) | 2.4 +/− 0.6 | 8.8 +/− 6.4 | 6.1 +/− 5.5 |
| (Cfu/□g DNA) | 4.6 × 10⁴ +/− 1.2 × 10⁴ | 4.3 × 10³ +/− 3.1 × 10³ | 1.6 × 10⁴ +/− 1.8 × 10² |

*Cell density in the preserved cells was 1/10 of that in fresh concentrates. These cells were transformed immediately after drying and they were not concentrated before electroporation.

Electroporation survival in the preserved cells was lower than in the fresh cells (12.2% or 35.7% versus 46.2%). In contrast, electroporation efficiency in the preserved cells was higher than in the fresh cells (6.1% or 8.8% versus 2.4%).

Electroporation survival in preserved control cells was lower than in preserved loaded cells. Electroporation efficiencies were comparable in unloaded and loaded preserved cells (6.1% versus 8.8%).

Based on the data from this example, the following conclusions were made:
1. Commercial electrocompetent E. coli cells were successfully preserved by the foam formation process.
2. Loading with MAG increased stability of the preserved cells at 4° C.
3. Electroporation survival was higher in fresh cells than in the preserved cells.
4. In preserved cells, electroporation survival was higher in loaded cells compared to unloaded control.
5. Electroporation efficiency was higher in the preserved cells compared to that in the fresh cells.
6. There was no significant difference in electroporation efficiency in MAG-loaded preserved cells versus unloaded preserved controls (MAG was slightly inhibitory).

### EXAMPLE 4

Chemically competent E. coli MM294 cells were prepared and preserved by foam formation as described previously. Preservation yield and stability of the preserved cells are described in Table 6.

TABLE 6

| | Preservation Yield (Day 0) | | Stability (290 Days at 4° C.) | |
|---|---|---|---|---|
| Cells | Cfu/ml | % | Cfu/ml | % |
| Fresh | 1.6 × 10¹⁰ +/− 6.8 × 10⁹ | N/A | N/A | N/A |
| Preserved | 5.1 × 10⁷ +/− 4.0 × 10⁷ | 0.3 +/− 0.3 | 1.1 × 10⁷ +/− 4.5 × 10⁶ | 0.07 +/− 0.03 |

Chemically competent E. coli MM294 cells were preserved at low yield (0.3%). The cells were harvested in early logarithmic stage and were not loaded with MAG or any other sugar derivative to enhance bacterial desiccation tolerance and the subsequent preservation survival.

The preserved cells were relatively stable at 4° C. After 290 days of storage, the observed loss in viability was less than 1 Log (Table 6).

Transformation efficiencies of the fresh and the preserved chemically competent cells are presented in Table 7.

TABLE 7
Transformation Efficiency

| | Day 0 | | 290 Days at 4° C. | |
|---|---|---|---|---|
| Cells | Cfu/ml | 10⁻³% | Cfu/ml | 10⁻³% |
| Fresh | 9.7 × 10⁵ +/− 1.9 × 10⁵ | 6.1 +/− 1.1 | N/A | N/A |
| Preserved | 6.7 × 10² +/− 2.4 × 10² | 1.3 +/− 0.5 | 1.6 × 10² +/− 5.0 × 10¹ | 1.5 +/0.5 |

Transformation efficiency in the preserved cells was somewhat lower compared to the efficiency of the fresh cells (Table 7). Preservation of the cells by foam formation did not compromise bacterial competence in any significant fashion. Transformation efficiency of the preserved cells stored for 290 days at 4° C. was comparable to the efficiency immediately after drying.

Based on the data of this example, the following conclusions were reached:
1. Chemically competent E. coli cells could be successfully preserved by foam formation.
2. Preservation yield was low because the cells were harvested in early logarithmic phase when they likely had no internal desiccation protectants.
3. The cells remained relatively stable upon prolonged storage at 4° C.
4. The preserved cells remained competent after preservation.
5. The competence function was not compromised during storage at 4° C.

### EXAMPLE 5

Accumulation of MAG in vitro enhances preservation survival of logarithmic E. coli cells. Preparation of electrocompetent cells routinely requires harvesting bacterial cultures in late logarithmic growth stage. In contrast, the cells must be harvested in an early or medium logarithmic growth stage for preparation of chemically competent cells.

Late logarithmic cells are more tolerant to desiccation than early logarithmic cells. We found that desiccation tolerance of logarithmic E. coli could be significantly enhanced by loading the cells with MAG. We also found that efficiency of loading could be influenced by the following parameters:
1. Choice of carbohydrate
2. Loading temperature
3. Loading time
4. Growth stage of the cells

Accumulation of non-metabolizable glucose analogs enhances preservation of logarithmic E. coli cells.

Bacterial culture was grown until OD₅₅₀=0.5 was reached (mid-log growth stage). The cell concentrates were prepared as described previously. Control cells, which were not loaded, were incubated on ice for 30 minutes. Cell concentrates were incubated with different amounts (0.15-1%, 5%, 10% and 15%) of glucose and glucose nonmetabolizable analogs at 37° C. for 30 minutes (loading). These mixtures were preserved as described previously.

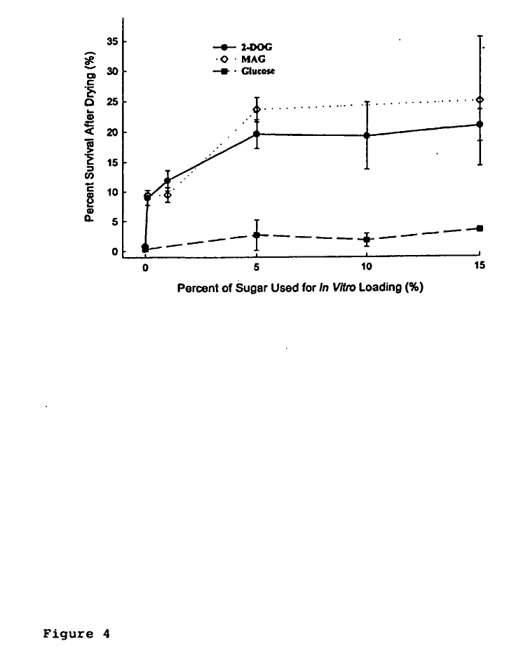

Mid-logarithmic cells loaded with 0.1%/-1% and 5% of MAG survived drying at 9%, 12% and at 24%, respectively. When cells were loaded with 0.1-1%, 5%, 10% and 15% of 2-DOG, bacteria survived drying at 9-12%, 20%, 16%, and 20%, respectively (FIG. 4). Loading with more than 5% of either MAG or 2-DOG in vitro did not improve drying survival compared to the survival obtained when 5% of either compound was used. Control cells, which were not loaded, survived drying at <1%.

In contrast to its nonmetabolizable analogs MAG or 2-DOG, glucose had no protective effect on preservation survival of logarithmic cells. Mid-logarithmic cells loaded in vitro with up to 15% of glucose survived drying at 1-3% (FIG. 4). There was no significant difference in survival when different amounts of glucose were used for loading. Because glucose was used at concentrations significantly higher than physiological, likely only a portion of the sugar was metabolized. Remaining glucose had no immediate damaging effect on preservation survival. However, because of its high reducing activity, glucose could be detrimental to cells due to a non-enzymatic browning, including the Mallard's reaction.

Based on the data of this example, the following conclusions were made:
1. Incubation of mid-logarithmic E. coli cells with 5% of MAG or 2-DOG prior to drying enhanced preservation survival of bacteria.
2. Incubation with more than 5% of nonmetabolizable sugar analogs had no additional effect on preservation survival.
3. Accumulation of glucose in the cells had no protective effect against desiccation stress during preservation.

### EXAMPLE 6

Accumulation of MAG in the cells prior to drying enhances preservation of logarithmic E. coli cells. Two cell concentrates (harvested at mid-log growth stage) were prepared as described previously. To induce uptake of MAG, 0.5% glucose was added to the growth medium (“induced culture”). Both concentrates were incubated with 10% MAG at 37° C. for 30 minutes (loading). When loading was completed, one concentrate was diluted 100-fold in PBS buffer and incubated at 37° C. for an additional 30 minutes to induce expulsion of the pre-accumulated MAG. Both concentrates were preserved as described previously.

Cell density in the concentrates was 4.7×10⁸ cfu/ml. Preservation yield in loaded concentrate, which was not subsequently diluted, was 57% (2.7×10⁸ cfu/ml). In contrast, in the concentrate, which was diluted to remove MAG pre-accumulated in the cells, preservation yield was only 3.8% (1.8×10⁷ cfu/ml). Cells loaded with MAG were stable at room temperature for extended periods of time (i.e. stability after 12 days was 52%, 2.5×10⁸ cfu/ml). Cells, which were depleted from pre-accumulated MAG by expulsion, and preserved at low yield, remained at 2.4% (1.1×10⁷ cfu/ml).

Based on the data of this example, the following conclusions were made:
1. Accumulation of non-metabolizable carbohydrate analogs, such as MAG, prior to drying enhances preservation survival of mid-log cells.
2. Removal of the accumulated sugar by expulsion results in sensitivity to desiccation and low preservation yield.

### EXAMPLE 7

Mid-logarithmic culture was prepared as described previously. 0.5% glucose was added to the growth medium (“induced cells”). The culture was concentrated 10-fold and the concentrates were incubated for 30 minutes with 10% MAG at different temperatures (0, 20, 40, and 60° C.). The mixtures were preserved as described previously.

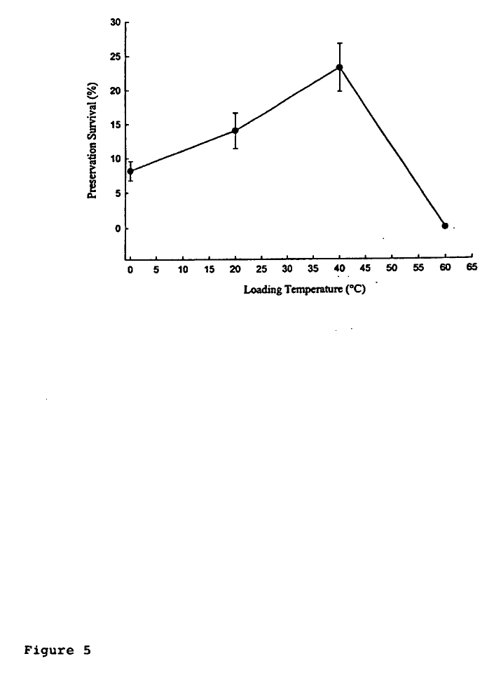

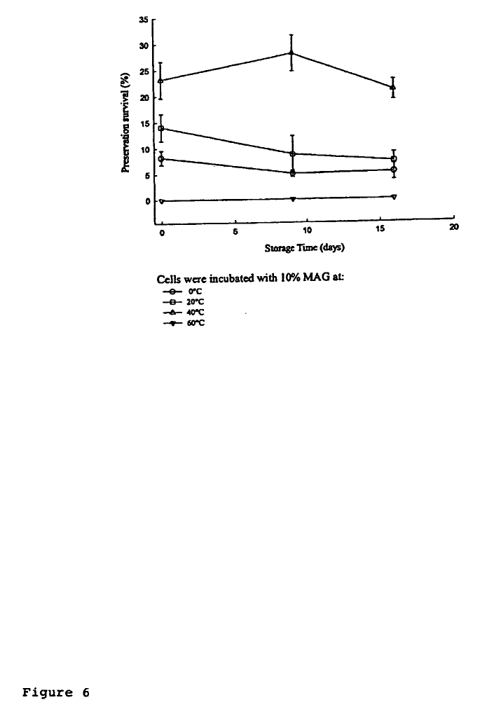

Cell densities (cfu/ml) in mid-logarithmic E. coli culture were 1.6×10⁸ cfu/ml. Cell density in the concentrate was 1.9×10⁹ cfu/ml. Preservation yield and stability of E. coli cells incubated with 10% MAG at different temperatures are shown in Table 8 and in FIGS. 5 and 6.

TABLE 8

| | Preservation Yield | | Stability (9 Days at RT°) | | Stability (16 Days at RT°) | |
|---|---|---|---|---|---|---|
| Sample | | % | | % | | % |
| Description | Cfu/ml | Survival | Cfu/ml | Survival | Cfu/ml | Survival |
| Loaded at | 1.6 × 10⁸ +/− | 8.2 +/− 1.4 | 9.4 × 10⁷ +/− | 5.0 +/− 0.6 | 9.9 × 10⁷ +/− | 5.2 +/− 1.5 |
| 0° C. | 2.7 × 10⁷ | | 1.2 × 10⁷ | | 2.8 × 10⁷ | |
| Loaded at | 2.7 × 10⁸ +/− | 14.1 +/− 2.6 | 1.6 × 10⁸ +/− | 8.7 +/− 0.6 | 1.4 × 10⁸ +/− | 7.3 +/− 1.7 |
| 20° C. | 4.9 × 10⁷ | | 6.7 × 10⁷ | | 3.2 × 10⁷ | |
| Loaded at | 4.4 × 10⁸ +/− | 23.2 +/− 3.5 | 5.3 × 10⁸ +/− | 28.1 +/− 3.5 | 3.9 × 10⁸ +/− | 21.0 +/− 1.9 |
| 40° C. | 6.6 × 10⁷ | | 6.6 × 10⁷ | | 3.7 × 10⁷ | |
| Loaded at | 1.4 × 10⁴ +/− | 0 | 1.2 × 10⁴ +/− | 0 | 1.3 × 10⁴ +/− | 0 |
| 60° C. | 1.4 × 10⁻³ | | 2.3 × 10³ | | 1.9 × 10³ | |

Logarithmic cells incubated with 10% MAG at 20° C. or at 40° C. preserved at higher yields and were more stable after 16 days of storage at room temperature compared to the cells incubated with MAG at 0° C. and at 60° C. (FIGS. 5 and 6). Cells incubated with MAG at 40° C. preserved at higher yield than cells incubated with MAG at 20° C. and remained more stable during storage. Incubation at 60° C. resulted in severe loss in bacterial viability.

Based on the data from this example, the following conclusions were reached:
1. Efficiency of loading of MAG into logarithmic E. coli cells is highly dependent on the incubation temperature.
2. Loading of mid-log E. coli cells with MAG was the most effective when the cells were incubated at 20°-40° C., preferentially closer to 40° C.

### EXAMPLE 8

Effect of incubation time on accumulation of MAG in logarithmic E. coli cells. Cell concentrates were prepared as described previously (0.5% glucose was added to the growth medium) and incubated with 10% MAG at 37° C. for 0, 2.5, 5, 15, 30, and 60 minutes. The mixtures were preserved as described previously.

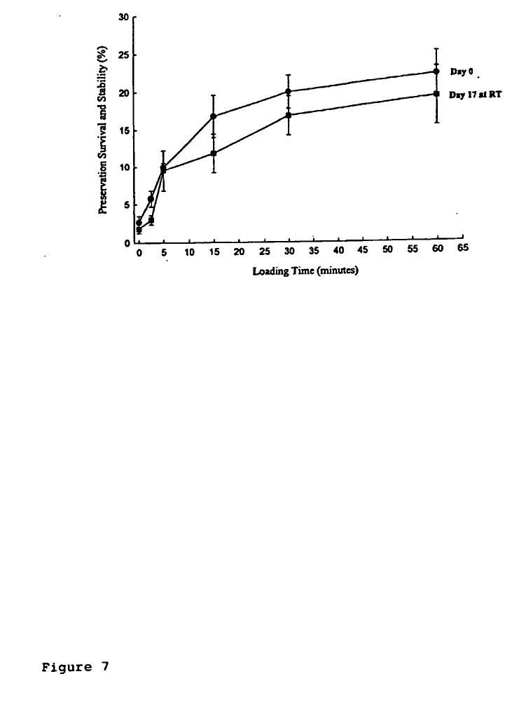

Preservation yield and stability of mid-logarithmic E. coli cells incubated with 10% MAG for 0-60 minutes are shown in Table 9 and on FIG. 7.

TABLE 9

| | Preservation Yield | | Stability (7 days at RT) | | Stability (17 days at RT) | |
|---|---|---|---|---|---|---|
| Sample | | % | | % | | % |
| Description | Cfu/ml | Survival | Cfu/ml | Survival | Cfu/ml | Survival |
| Incubated | 5.3 × 10⁷ +/− 1.7 × 10⁷ | 2.6 +/− 0.8 | 2.4 × 10⁷ +/− | 1.2 +/− 0.7 | 3.6 × 10⁷ +/− | 1.8 +/− 0.5 |
| for 0 min | | | 1.4 × 10⁷ | | 1.1 × 10⁷ | |
| Incubated | 1.2 × 10⁸ +/− 2.1 × 10⁷ | 5.8 +/− 1.1 | 9.6 × 10⁷ +/− | 4.9 +/− 0.8 | 5.9 × 10⁷ +/− | 2.9 +/− 0.6 |
| for 2.5 min | | | 1.5 × 10⁷ | | 1.2 × 10⁷ | |
| Incubated | 2.0 × 10⁸ +/− 1.1 × 10⁷ | 9.9 +/− 0.6 | 1.4 × 10⁸ +/− | 6.8 +/− 1.2 | 1.9 × 10⁸ +/− | 9.5 +/− 2.7 |
| for 5 min | | | 2.4 × 10⁸ | | 5.4 × 10⁷ | |
| Incubated | 3.4 × 10⁸ +/− 5.6 × 10⁷ | 16.8 +/− 2.8 | 2.1 × 10⁸ +/− | 10.4 +/− 0.4 | 2.4 × 10⁸ +/− | 11.8 +/− 2.6 |
| for 15 min | | | 8.3 × 10⁶ | | 5.2 × 10⁷ | |
| Incubated | 4.0 × 10⁸ +/− 4.4 × 10⁷ | 19.9 +/− 2.2 | 2.6 × 10⁸ +/− | 13.0 +/− 2.7 | 3.4 × 10⁸ +/− | 16.8 +/− 2.6 |
| for 30 min | | | 5.4 × 10⁷ | | 5.2 × 10⁷ | |
| Incubated | 4.4 × 10⁸ +/− 6.1 × 10⁷ | 22.2 +/− 3.0 | 3.3 × 10⁸ +/− | 16.3 +/− 1.9 | 3.9 × 10⁸ +/− | 19.3 +/− 3.9 |
| for 60 min | | | 3.7 × 10⁷ | | 7.7 × 10⁷ | |

Logarithmic cells incubated with MAG for 2.5-60 minutes preserved at higher yields than cells incubated with MAG for 0-2.5 minutes. Preservation survival of mid-logarithmic E. coli cells was directly proportional to the time of incubation with MAG (FIG. 7). After 17 days of storage at RT, the cells incubated with MAG for at least 2.5 minutes were more stable than the cells incubated with MAG for less than 2.5 minutes.

The following conclusions were reached, based on the data of this example:
1. Incubation with MAG for at least 2.5 minutes enhances preservation survival of mid-log E. coli cells. Loading for at least 5 minutes enhanced stability of the preserved cells.
2. Preservation yield and stability of the preserved cells are directly proportional to the length of incubation with MAG.

### EXAMPLE 9

Effect of growth stage on accumulation of MAG in E. coli cells. E. coli cultures were grown in L-broth with 0.5% glucose as described previously and harvested at OD₅₅₀=0.1 (early-log), 0.5 (mid-log), 1.0 (late-log) and 2.0 (stationary cells). The cells in each culture were concentrated and split in two aliquots. One aliquot of each concentrate was incubated with 10% MAG at 37° C. for 30 minutes (“loaded”). The second aliquot was not loaded (controls). Unloaded controls and loaded cells were preserved as described previously.

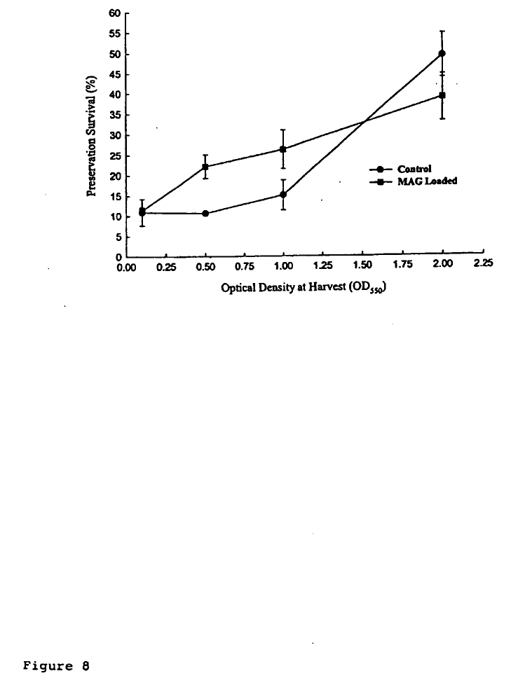

Cell densities at different ODs were the following: 4.2×10⁷ cfu/ml (OD₅₅₀=0.1), 1.7×10⁸ cfu/ml (OD₅₅₀=0.5), 8.0×10⁸ cfu/ml (OD₅₅₀=1.0) and 1.3×10⁹ cfu/ml (OD₅₅₀=2.0). Preservation yield of E. coli harvested at different growth stages and loaded with 10% MAG is shown in Table 10 and on FIG. 8.

TABLE 10

| | Preservation Yield (Day 0) | | Stability (Day 7 at RT) | | Stability (Day 14) | |
|---|---|---|---|---|---|---|
| Sample | | % | | % | | % |
| Description | Cfu/ml | Survival | Cfu/ml | Survival | Cfu/ml | Survival |
| OD = 0.1, | 1.6 × 10⁷ +/− | 10.9 +/− 3.3 | 8.9 × 10⁶ +/− | 6.1 +/− 1.0 | 1.1 × 10⁷ +/− | 7.0 +/− 1.9 |
| control | 4.8 × 10⁶ | | 1.4 × 10⁶ | | 2.8 × 10⁶ | |
| OD = 0.1, | 1.6 × 10⁷ +/− | 11.3 +/− 0.9 | 1.2 × 10⁷ +/− | 8.2 +/− 0.8 | 1.5 × 10⁷ +/− | 9.9 +/− 2.2 |
| loaded | 1.3 × 10⁶ | | 1.2 × 10⁶ | | 3.2 × 10⁶ | |
| OD = 0.5, | 2.6 × 10⁸ +/− | 10.7 +/− 0.6 | 2.5 × 10⁸ +/− | 10.0 +/− 1.3 | 1.8 × 10⁸ +/− | 7.3 +/− 0.3 |
| control | 1.5 × 10⁷ | | 3.3 × 10⁷ | | 8.3 × 10⁵ | |
| OD = 0.5, | 5.5 × 10⁸ +/− | 22.2 +/− 2.9 | 5.3 × 10⁸ +/− | 21.6 +/− 4.7 | 4.3 × 10⁸ +/− | 17.4 +/− 1.2 |
| loaded | 7.2 × 10⁷ | | 1.2 × 10⁸ | | 2.9 × 10⁷ | |
| OD = 1.0, | 1.3 × 10⁹ +/− | 15.1 +/− 3.7 | 1.4 × 10⁹ +/− | 16.2 +/− 2.8 | 1.6 × 10⁹ +/− | 18.4 +/− 4.6 |
| control | 3.3 × 10⁸ | | 2.5 × 10⁸ | | 4.0 × 10⁸ | |
| OD = 1.0, | 2.3 × 10⁹ +/− | 26.3 +/− 4.7 | 3.2 × 10⁹ +/− | 36.1 +/− 7.5 | 2.9 × 10⁹ +/− | 33.0 +/− 5.7 |
| loaded | 4.2 × 10⁸ | | 6.5 × 10⁸ | | 4.9 × 10⁸ | |
| OD = 2.0, | 3.5 × 10⁹ +/− | 49 +/− 5.4 | 4.1 × 10⁹ +/− | 57.5 +/− 6.8 | 4.3 × 10⁹ +/− | 60.3 +/− 11.5 |
| control | 3.8 × 10⁸ | | 4.8 × 10⁸ | | 8.2 × 10⁸ | |
| OD = 2.0, | 2.8 × 10⁹ +/− | 38.8 +/− 5.8 | 2.1 × 10⁹ +/− | 29.9 +/− 3.6 | 2.5 × 10⁹ +/− | 35.3 +/− 7.5 |
| loaded | 4.1 × 10⁸ | | 2.6 × 10⁸ | | 5.3 × 10⁸ | |

No significant difference in preservation survival between loaded and unloaded cells were found when the cells were harvested in early logarithmic stage (at OD₀₀₅=0.1) before loading with 10% MAG. In contrast, the cells harvested in mid-log or late-log growth stage and loaded with MAG preserved at higher yield and were significantly more stable than unloaded controls. The stationary cells, loaded or unloaded, preserved at higher yield than loaded cells. High preservation survival in stationary cells could be attributed to the presence of intracellular protective compounds.

The importance of the growth stage for efficient loading of MAG is clearly illustrated on FIG. 8. Regardless of a mechanism of MAG accumulation, significant difference in preservation survival and stability between control and loaded cells was obtained only in early- and mid-logarithmic cells suggesting that the loading process was growth-stage dependent.

Based on the data from this example, the following conclusions were reached:
1. Efficiency of loading the cells with MAG is highly dependent upon growth stage of bacteria.
2. In vitro loading with MAG significantly increased preservation yield and stability in mid-log and late-log cells.
3. Loading with MAG resulted in a decrease in preservation survival in stationary cells.
4. Early-log cells preserved at lower yield and were less stable compared to the cells in later growth stage.
5. Stationary cells were the most stable regardless of loading.

### EXAMPLE 10

Chemically competent E. coli XL10-Gold cells (1.5 L; Stratagene) in the form of a bacterial pellet were preserved using foam formation. The cells were concentrated 50-fold by resuspending the pellets in 30 ml of transformation buffer (Stratagene) and processed in the following manner.

This example provides method steps which differ than those previously disclosed. Therefore, the methods of this example are included herein in their entirety.

Methods

(1) Cell density: To determine the cell density in the material, the cells (2×0.1 ml) were diluted in SM buffer (Stratagene) and plated in duplicate (0.1 ml) on L-agar plates.

(2) Transformation of fresh cells: 100 [symbol] l of cells+0.1 ng or 0.01 ng of pUC18 plasmid DNA (Stratagene) were mixed and incubated on ice for 30 minutes. When 30 minutes elapsed, the mixtures were transferred in a 42° C. water bath and subjected to a “heat shock” for 30 seconds. The mixtures were then incubated on ice for 2 minutes and 1 ml of NZY medium (Stratagene) was added. The mixtures were incubated at 37° C. for 60 minutes with shaking (200 rpm). The cells were plated on L-agar plates with 100 [symbol] g/mi ampicilin (50, 100, 200, and 400 [symbol] l aliquots of undiluted mixtures) for determination of transformation efficiency. The plates were incubated overnight at 37° C.

(3) Freezing of the fresh cells: 10×0.15 ml of the original concentrate were aliquoted in Eppendorf tubes, mixed with 10% DMSO, and frozen at −80° C. Preservation yield and stability in the frozen cells was determined after storage at −80° C. for 3 and 7 days.

(4) Preservation of electrocompetent cells by foam formation: The cells were split in two aliquots and mixed either with preservation solution “A” (22.5% 2:1 sucrose:raffinose) or with solution “B”(22.5% 4:1 sucrose:MAG) 43×0.5 ml of the mixture “A”was aliquoted in 43 sterile 5 ml vials. Similarly, 9×0.5 ml of the mixture “B” was aliquoted in 9 sterile 5 ml vials. Vials containing the two preservation mixtures were kept on ice until preservation. Bacterial survival in the preservation mixtures was determined by plating appropriate dilutions on L-agar plates. The plates were incubated overnight at 37° C.

(5) Drying: Preservation mixtures were dried overnight by foam formation. Vials containing the mixture “A” were split in two groups and stored at 4° C. and at room temperature (RT). Vials containing the mixture “B” were stored at RT only. Stability of the preserved material was initially evaluated after 7 days of storage and will be monitored over time.

(6) Determination of preservation yield: The preserved cells were rehydrated at RT with 0.5 ml of transformation buffer (STB) (Stratagene) or transformation buffer (UTB) (Universal Preservation Technologies). Stratagene's transformation buffer is available commercially. UTB buffer comprises: 10 mM MOPS, 75 mM calcium chloride, 10 mM rubidium chloride and 10% sucrose. Appropriate dilutions were plated on L-agar plates and the plates were incubated at 37° C. overnight.

(7) Transformation of the preserved cells: The preserved cells were transformed in the same manner as the fresh cells.

Results

Chemically competent E. coli XL10-Gold cells (Stratagene) were preserved by the foam formation and by freezing at −80° C. The cells were preserved in two different solutions and rehydrated with two different buffers in order to determine possible effects of these parameters on the preservation yield. Preservation yield and stability of E. coli XL10-Gold cells were determined and presented in Table 11.

TABLE 11

| | Preservation yield (Day 0) | | Stability (Day 7) | | | |
|---|---|---|---|---|---|---|
| Cells | Cfu/ml | % | Cfu/ml | | % | |
| Fresh | 1.0 × 10¹⁰ +/− | 100 | N/A | | N/A | |
| | 2.6 × 10⁸ | | | | | |
| Frozen | 2.5 × 10⁹ +/− | 24.1 +/− 1.4* | 2.5 × 10⁹ +/− 1.1 × 10⁸ | | 24.4 +/− 1.0 | |
| | 1.5 × 10⁸* | | | | | |
| Foam | | | | | | |
| Formation | | | | | | |
| Preserved | | | | | | |
| PS “A”, | 9.5 × 10⁷ +/− | 0.9 +/− 0.2 | 4° C. | RT | 4° C. | RT |
| UTB | 2.1 × 10⁷ | | 3.9 × 10⁷ +/− 1.0 × 10⁷ | 3.5 × 10⁷ | 0.38 +/− | 0.34 |
| | | | | +/− | 0.10 | +/− |
| | | | | 2.3 × 10⁷ | | 0.22 |
| PS “B”, | 3.4 × 10⁶ +/− | 0.03 +/− 0.02 | 4° C. | RT | 4° C. | RT |
| UTB | 1.9 × 10⁶ | | N/A | 1.8 × 10⁵ | N/A | <0.01 |
| | | | | +/− | | |
| | | | | 9.3 × 10⁴ | | |

| | | | | |
|---|---|---|---|---|
| PS “A”, | 1.1 × 10⁸ +/− | 1.1 +/− 0.3 | N/D | N/D |
| STB | 2.8 × 10⁷ | | | |
| PS “B”, | 2.0 × 10⁶ +/− | 0.02 +/− 0.00 | N/D | N/D |
| STB | 2.5 × 10⁵ | | | |

PS, preservation solution
UTB, transformation buffer (Universal Preservation Technologies)
STB, transformation buffer (Stratagene)
*Day 1 at −80° C.
N/A, not applicable
N/D, not determined

Preservation yield of cells dried by foam formation were approximately 0.9% (Table 11). The yield was significantly lower than preservation yield of the frozen cells. Preservation yield in the cells preserved in solution “A” and rehydrated with UTB was comparable to that in the cells preserved in the same solution and rehydrated with STB. Similar findings were found for the cells preserved in solution “B”. However, overall preservation yield was significantly higher (>30-fold) when the cells were preserved in solution “A” compared to that obtained with solution “B” regardless of the rehydration buffer.

After 7 days of storage at 4° C. and at RT, loss in viability of about ½ Log was observed in the material preserved in solution “A”. Also, a loss of about 1 Log was observed in the material preserved in solution “B”. There was no significant difference in stability of the material preserved in either solution at the two temperatures. No loss in viability was observed in frozen cells after 7 days at −80° C.

Transformation efficiency of the preserved material is presented in Table 12. The cells were transformed as described in the methods section of this example.

TABLE 12

| | Transformation |
|---|---|
| | Efficiency* |
| Sample Description | (Cfu/□g pUC18) |
| Fresh | 7.4 × 10⁶ +/− 4.9 × 10⁵ |
| Frozen | 3.1 × 10⁶ +/− 3.7 × 10⁵ |
| Foam formation preserved (Day 0) | |
| PS “A”, UTB | 7.6 × 10⁶ +/− 1.2 × 10⁶ |
| PS “B”, UTB | 8.0 × 10⁵ +/− 1.2 × 10⁵ |
| PS “A”, STB | 5.9 × 10⁵ +/− 8.8 × 10⁴ |
| PS “B”, STB | 3.8 × 10⁴ +/− 1.7 × 10⁴ |
| Frozen (Day 4 at −80° C.) | 1.1 × 10⁶** |
| Foam formation preserved (Day 7) | |
| PS “A”, UTB, stored at 4° C. | 3.5 × 10⁶ +/− 9.2 × 10⁵ |
| PS “A”, UTB, stored at RT | 4.0 × 10⁶ +/− 4.6 × 10⁵ |
| PS “B”, UTB, stored at RT | 1.0 × 10⁶ +/− 3.6 × 10⁵ |
| PS “A”, STB | N/D |
| PS “B”, STB | N/D |

*Transformation efficiencies reported were obtained with 0.1 ng of pUC18. No significant difference was observed when 0.01 ng of pUC18 was used instead of 0.1 ng.
**Only one sample was plated (0.4 ml) and a standard deviation could not be determined.
N/D, not determined.

Transformation efficiency in the cells preserved by foam formation was comparable to that in the fresh and frozen cells (Table 12). The cells preserved in solution “A” and rehydrated with transformation buffer (Universal Preservation Technologies) had the highest efficiency after drying.

Storage at 4° C. or at RT did not compromise transformation efficiency of the preserved cells. There was no difference in the efficiency of the preserved cells stored for 7 days compared to the cells immediately after drying.

The feasibility of preservation of chemically competent E. coli XL10-Gold cells was clearly demonstrated by the results discussed above. Differences in yield were observed for the cells preserved in the two different preservation solutions (“A” and “B”) evaluated in this experiment. There was no difference in the recovery of the cells preserved in solution “A” or “B” with respect to which rehydration solution was used (UTB or STB buffer). Overall, preservation survival in the cells dried by the foam formation was lower than for frozen cells (0.9-1.1% versus 24.1%).

Chemically competent cells preserved in this example were harvested in a mid-logarithmic growth stage. Some initial loss in viability of the preserved cells was observed after 7 days of storage at either RT or 4° C. It is not unusual for cells preserved in logarithmic growth stage without desiccation protectants, like chemically competent cells used in the present example, to lose some stability after initial storage. Other protective solutions can be used to modulate stability of the preserved material in this example.

Transformation efficiency in the preserved cells was comparable to that in the fresh and frozen cells. In contrast to preservation survival, where preservation solution was the factor critical to the recovery of the preserved cells, both preservation and rehydration solutions were critical parameters affecting the efficiency of the preserved cells. In addition, the cells rehydrated with buffer UTB transformed at efficiencies at least 10-fold higher than the cells rehydrated in buffer STB. Combination of preservation solution “A” and rehydration buffer (Universal Preservation Technologies) resulted in the cells which had the maximal transformation efficiency (7.6×10⁶ cfu/ [symbol] g) after drying.

Transformation efficiency in the preserved cells remained unchanged after storage for 7 days at 4° C. or at RT. In conclusion, chemically competent E. coli XL10-Gold cells were successfully preserved by foam formation and remained fully competent after preservation and a short-term storage.

### EXAMPLE 11

Electrocompetent E coli XL1Blue cells (3 L; Stratagene) in the form of a bacterial pellet were preserved by the foam formation process. The cells were concentrated 100-fold by resuspending the pellets in 30 ml concentrating solution (Universal Preservation Technologies) and processed in the following manner.

This example provides method steps which differ than those previously disclosed. Therefore, the methods of this example are included in their entirety.

Methods

(1) Cell density: To determine the cell density in the material, the cells (2×0.1 ml) were diluted in SM buffer (Stratagene) and plated in duplicate (0.1 ml) on L-agar plates.

(2) Electroporation of fresh cells: 40 [symbol] l of cells+0.1 ng of pUC18 plasmid DNA (Stratagene) were mixed, transferred into 0.1 cm BioRad cuvette (Stratagene) and electroporated at E=1.7 kV, R=200 Ohm, C=25 [symbol] F [symbol] [symbol] =4-5 msec). After the pulse, 1 ml of SOC medium (Stratagene) was added to the cells and the mixture was incubated at 37° C. for 60 minutes with shaking (250 rpm). The cells were plated on L-agar plates with no antibiotic (for determination of electroporation survival) and on L-agar plates with 100 [symbol] g/ml ampicilin (for determination of electroporation efficiency). The plates were incubated overnight at 37° C.

(3) Freezing of the fresh cells: 10×0.1 ml of the original concentrate was aliquoted in Eppendorf tubes, mixed with 10% DMSO, and frozen at −80° C. Preservation yield and stability in the frozen cells were determined after storage at −80° C. for 1 and 12 days.

(4) Preservation of electrocompetent cells by foam formation: 3.6 ml of the cells were removed from the 30 ml of the material, centrifuged, and the pellet was resuspended in preservation solution #1 (Universal Preservation Technologies). The mixture was aliquoted in 16×1.2 ml vials (0.2 ml per vial). Similarly, 24.8 ml of the remaining cells were centrifuged and the pellet was resuspended in preservation solution #2 (Universal Preservation Technologies). The mixture was aliquoted in 16×1.2 ml vials (0.2 ml per vial) and in 43×5 ml vials (0.5 ml per vial). Vials containing the two preservation mixtures were kept on ice until preservation. Bacterial survival in preservation mixtures was determined by plating appropriate dilutions on L-agar plates. The plates were incubated overnight at 37° C.

(5) Drying: Preservation mixtures were dried overnight by foam formation. Vials containing the preserved cells were stored at 4° C. and at room temperature (RT). The 1.2 ml vials were stored at 4° C. The 5 ml vials were divided in two groups and stored at RT and at 4° C. Stability of the preserved material was initially evaluated after 11 days of storage.

(6) Determination of preservation yield: The preserved cells (1.2 ml vials, preservation solutions 1&2; 5 ml vials, preservation solution 2) were rehydrated at room temperature (RT) in solution “A” (Universal Preservation Technologies). 0.5 ml of the solution was added to rehydrate the cells in 5 ml vials, 0.2 ml was used for rehydration of the cells in 1.2 ml vials. In addition, several 5 ml vials (material preserved in solution 2) were rehydrated with solution “B” (Universal Preservation Technologies). Appropriate dilutions were plated on L-agar plates and the plates were incubated at 37° C. overnight.

(7) Electroporation of preserved cells: The preserved cells were electroporated in the same manner as the fresh cells.

Results

Electrocompetent E. coli XL1Blue cells (Stratagene) were preserved by foam formation and by freezing at −80° C. The cells were preserved in two different solutions, in two vial sizes, and rehydrated with two different solutions in order to determine a possible effect of these parameters on preservation yield. Preservation yield and stability of the E. coli XL1Blue cells were determined and presented in Table 13.

TABLE 13

| | | | Stability (Day 12 frozen; Day 11 Foam | | | |
|---|---|---|---|---|---|---|
| | Preservation yield (Day 0) | | Formation Preserved) | | | |
| Cells | Cfu/ml | % | Cfu/ml | | % | |
| Fresh | 6.5 × 10⁹ +/− 1.8 × 10⁹ | 100 | N/A | | N/A | |
| Frozen | N/D | N/D | 1.2 × 10⁹ +/− 1.1 × 10⁸ | | 18.6 +/− 1.7 | |
| Foam | | | | | | |
| Formation | | | | | | |
| Preserved | | | | | | |
| 1.2 ml vial, | 2.6 × 10⁸ +/− 1.7 × 10⁷ | 4.0 +/− 0.3 | N/D | | N/D | |
| PS#1, RS “A” | | | | | | |
| 1.2 ml vial, | 2.6 × 10⁸ +/− 3.3 × 10⁷ | 4.0 +/− 0.5 | N/D | | N/D | |
| PS#2, RS “A” | | | | | | |
| 5 ml vial, | 3.5 × 10⁸ +/− 5.3 × 10⁷ | 5.3 +/− 0.8 | N/D | | N/D | |
| PS#2, RS “A” | | | N/D | | N/D | |
| 5 ml vial, | 5.1 × 10⁸ +/− 1.5 × 10⁸ | 7.9 +/− 2.3 | 4° C. | RT | 4° C. | RT |
| PS#2, RS “B” | | | 4.4 × 10⁸ +/− 6.6 × 10⁷ | 3.2 × 10⁸ +/− 6.1 × 10⁷ | 6.8 +/− 1.0 | 5.1 +/− 1.0 |

PS, preservation solution
RS, rehydration solution
N/A, not applicable
N/D, not determined

Preservation yield of foam formation dried cells was low, ranging from 4-8% (Table 13). No significant difference was found in preservation yield when the cells were preserved in two different solutions (PS#1 and PS#2) or in different size vials (1.2 ml or 5 ml) and rehydrated in solution “A” (Universal Preservation Technologies). Preservation yield was slightly higher when the cells preserved in 5 ml vials were rehydrated in solution “B” (Universal Preservation Technologies).

The preserved cells were stable for 11 days at 4° C. and at RT. There was no significant difference in the stability of the preserved material at the two temperatures.

Electroporation survival and efficiency of the preserved material are presented in Table 14. The cells were electroporated by the protocol as described previously in the methods section of this example.

TABLE 14

| | Electroporation | Electroporation |
|---|---|---|
| | Survival | Efficiency |
| Sample Description | (%) | (Cfu/□g pUC18) |
| Fresh | 19.3 +/2.1 | 8.7 × 10⁷ +/− 5.6 × 10⁶ |
| Frozen (Day 1 at −80° C.) | N/D | 1.8 × 10⁶ +/− 3.5 × 10⁵ |
| Frozen (Day 12 at −80° C.) | 19.7 +/− 1.6 | 1.2 × 10⁷ +/− 2.8 × 10⁶ |

Foam Formation Preserved (Day 0)

| | | |
|---|---|---|
| 1.2 ml vial, PS#1, RS “A” | 18.1 +/− 5.9 | 2.5 × 10⁵ +/− 1.3 × 10⁵ |
| 1.2 ml vial, PS#2, RS “A” | 24.1 +/− 7.8 | 9.5 × 10⁵ +/− 5.2 × 10⁵ |
| 5 ml vial, PS#2, RS “A” | 16.0 +/6.5 | 2.0 × 10⁵ +/− 1.6 × 10⁵ |
| 5 ml vial, PS#2, RS “B” | 5.6 +/0.4 | 7.3 × 10⁵ +/− 3.3 × 10⁵ |

Foam Formation Preserved (Day 11 at 4° C.)

| | | |
|---|---|---|
| 5 ml vial, PS#2, RS “B” | 8.9 +/− 2.0 | 1.0 × 10⁶ +/− 1.7 × 10⁵ |

Foam Formation Preserved (Day 11 at RT)

| | | |
|---|---|---|
| 5 ml vial, PS#2, RS “B” | 7.8 +/− 1.7 | 6.8 × 10⁵ +/− 1.7 × 10⁵ |

N/D, not determined

Except for samples preserved in solution #2 (PS#2) in 5 ml vials and rehydrated in solution “B”, there was no significant difference in electroporation survival of the preserved E. coli XL1Blue cells at Day 0 compared to the fresh cells (Table 14). In addition, there was no significant difference in electroporation survival with respect to a vial size in the cells preserved in solution #1. The cells preserved in solution #2 in 5 ml vials and rehydrated in solution “B” survived electroporation at a lower rate than the cells rehydrated in solution “A”.

Electroporation survival in the stored cells was determined for the sample preserved in solution #2 in 5 ml vials and rehydrated in solution “B”. There was no difference in electroporation survival after storage at 4° C. or at RT for 11 days compared to that at Day 0 (Table 2).

Electroporation efficiency in the preserved cells was lower compared to that in the fresh or frozen cells (Table 14). When the cells were preserved in 5 ml vials and rehydrated in solution “B”, electroporation efficiency was higher than in the cells preserved under identical conditions and rehydrated in solution “A”.

Storage at 4° C. or at RT did not compromise electroporation efficiency of the preserved cells. There was no difference in electroporation efficiency of the preserved cells stored for 11 days compared to the cells immediately after drying.

Based on the data from this example, the following conclusions were made:
1. The feasibility of preservation of electrocompetent E. coli XL1Blue cells was confirmed in the experiment discussed in this example.
2. With respect to formulation of preservation solution, no significant difference in the preservation yield was observed when the cells were preserved in two solutions evaluated in this experiment.
3. The preserved electrocompetent cells were stable at 4° C. and at RT for 12 days. No significant difference in stability was observed at the two temperatures.
4. Electroporation survival in the preserved cells was comparable to that in the fresh cells and was not compromised by storage at RT or at 4° C.
5. With the preservation and rehydration solutions used in this study, arcing was not a problem. Also, the formulations used for preservation and rehydration in this example resulted in an overall increase in the transformation efficiency of the preserved cells compared to the efficiency obtained in a similar experiment performed previously (7.3×10⁵ cfu/ [symbol] g pUC18 DNA compared to 5.0×10⁴ cfu/ [symbol] g).

Electroporation efficiency in the preserved cells obtained in the experiment presented in this report (7.3×10⁵ cfu/ [symbol] g) was slightly lower than in the frozen cells (1.8×10⁶ cfu/ [symbol] g).

In a separate study, we found that the concentration of sugars used in preservation and rehydration solutions has an inhibitory effect on electroporation efficiency when the cells were electroporated at a high voltage (1.7 kV). Therefore, to achieve the maximal efficiency of the cells, both preservation conditions (solutions to be used, drying protocols, rehydration solutions, etc.) and the conditions for electroporation (voltage, pulse duration, etc.) need to be optimized.

Although the invention has been described in detail for the purposes of illustration, it is understood that such detail is solely for that purpose, and variations can be made therein by those skilled in the art without departing from the spirit and scope of the invention which is defined by the following claims. All references referred to above are hereby incorporated by reference.

1. A method of preserving competent bacterial cells for storage at ambient temperatures, comprising:
incubating said cells with a nonmetabolizable and non-reducing carbohydrate analog, wherein said incubation results in accumulation of said analog in said cells; and
drying said cells by foam formation.

2. The method of claim 1, wherein said nonmetabolizable and non-reducing carbohydrate analog is α-methyl-glucoside (MAG) or 2-deoxyglucose (2-DOG).

3. The method of claim 1, wherein said cells are gram negative bacteria.

4. The method of claim 1, wherein said cells are Escherichia coli (E. coli).

5. The method of claim 1, wherein said cells are electrocompetent.

6. The method of claim 1, wherein said cells are chemically competent.

7. The chemically competent cells of claim 6, wherein competence is achieved by mixing said cells with CaCl₂ or RbCl.

8. The method of claim 1, further comprising rehydrating the dried cells by contacting said cells with a solution comprising at least one member of the group consisting of carbohydrates, mono-valent cations, divalent cations, organic buffers, and water.

9. The method of claim 8, wherein said carbohydrate is sucrose.

10. The method of claim 8, wherein said cations are calcium or rubidium.

11. The method of claim 1, wherein said carbohydrate analog is administered to said cells at concentrations of 0.1%-50% of total preservation solution.

12. The method of claim 1, wherein said carbohydrate analog is administered to said cells at concentrations of 5%-15% of total preservation solution.

13. The method of claim 1, wherein said logarithmic cells are mid-logarithmic cells.

14. The method of claim 1, wherein said logarithmic cells are late-logarithmic cells.

15. The method of claim 1, wherein said logarithmic cells are harvested at OD₅₅₀ between 0.1-2.0.

16. The method of claim 1, wherein said logarithmic cells are harvested at OD₅₅₀ between 0.3-1.0.

17. The method of claim 1, wherein said logarithmic cells are harvested at OD₅₅₀ at 0.5.

18. The method of claim 1, wherein said incubation is conducted at 0° C.-60° C.

19. The method of claim 1, wherein said incubation is conducted at 20° C.-40° C.

20. The method of claim 1, wherein said incubation is conducted at 30° C.-40° C.

21. The method of claim 1, wherein said incubation is conducted for 0-60 minutes.

22. The method of claim 1, wherein said incubation is conducted for 2.5-60 minutes.

23. The method of claim 1, wherein said incubation is conducted for 30-60 minutes.

24. The method of claim 1, wherein said carbohydrate analogs are actively transported into said logarithmic cells.

25. The method of claim 1, wherein said logarithmic cells are grown in the presence of glucose prior to preservation in order to prime said cells for active transport.

26. The method of claim 25, wherein said logarithmic cells are grown in growth solution comprising 0.001%-50% glucose.

27. The method of claim 25, wherein said logarithmic cells are grown in growth solution comprising 0.050%-5% glucose.
